(19) United States
(12) Patent Application Publication
(10) Pub. No.: US 2021/0402020 A1
(43) Pub. Date: Dec. 30, 2021

(54) PORTABLE STERILIZATON CONTAINER

(71) Applicant: Blue Horseshoe Manufacturing Inc., Winnipeg (CA)

(72) Inventor: Andrew J. Rhodes, St. Andrew's (CA)

(21) Appl. No.: 17/313,095

(22) Filed: May 6, 2021

(30) Foreign Application Priority Data
Jun. 24, 2020 (CA) 3084734

(51) Int. Cl. A61L 2/10; A61L 2/26

(52) U.S. Cl. CPC A61L 2/10; A61L 2/26; A61L 2202/121; A61L 2202/11; A61L 2202/122; A61L 2202/16

(57) ABSTRACT

A portable shipping container is converted for use in sterilizing articles and includes a wall separating the container into a control room at one end and a treatment room with each room having an access door. The treatment room has a plurality of portable replaceable racks on rollers for readily releasable coupling to a respective side wall and including rails on which articles to be sterilized can be suspended. Each sidewall and the roof has a plurality of elongate UVC illumination fixtures for generating UVC illumination and at least one of the light fixtures being mounted on the rack and an airflow fan system arranged to generate a circulation of air within the treatment room so as to cause movement by the air of the articles on the racks to ensure that the UVC illumination reaches all surfaces of the articles.

This invention relates to a portable sterilization container for receiving and sterilizing articles which could be infected by a pathogen such as a virus.

### BACKGROUND OF THE INVENTION

In recent years pathogens including insects such as bed bugs, bacteria and viruses have become more prevalent and more dangerous. Sterilization by heat and UV light is well known but up to now there has been no potable unit which can be moved to a required location and can treat all types of pathogens.

### SUMMARY OF THE INVENTION

According to the invention there is provided a portable container apparatus for use in sterilizing articles which could be infected with a pathogen comprising:

a portable shipping container having a roof, two side walls and a floor defining a portable structure which can be carried on a transport trailer;

the container being divided by a separating wall across the container into a control room at a first end and a treatment room at a second end;

at least one door providing access to the treatment room and at least one door providing access to the control room;

at least one rack having a plurality of rails on which articles to be sterilized can be suspended;

a plurality of light fixtures each arranged to generate UVC illumination;

some of the light fixtures being mounted at the roof for illuminating downwardly onto the articles on the rack;

some of the light fixtures being mounted at the side walls for illuminating inwardly into the interior of the treatment room onto the articles on the rack;

at least one of the light fixtures being mounted on the rack;

and an airflow generating fan construction arranged to generate a circulation of air within the treatment room so as to cause movement by the air of the articles on the rack to ensure that the UVC illumination reaches all surfaces of the articles.

According to one important optional feature there is provided a rack at each side wall with a center aisle between the racks.

According to one important optional feature there is provided the rack is removable for replacement by an alternative rack of different configuration and includes an electrical coupling connecting with each light fixture on the rack for common attachment to an outlet in the treatment room.

According to one important optional feature there is provided the rack is mounted on the side wall.

According to one important optional feature there is provided there are provided snap fasteners on the side wall for ready removal of the rack.

According to one important optional feature there is provided the rack includes a plurality of shelves.

According to one important optional feature there is provided the fan construction includes a first heater fan and a second airflow fan at an angle to the first fan so as to cause a circulation of the air. Preferably the first heater fan is arranged at the divider wall and the second airflow fan ais arranged at a corner between a side wall and an end wall.

According to one important optional feature there is provided there is provided an air evacuation unit connected between the interior of the treatment room and the exterior to rapidly remove heated air in the treatment room after the treatment is completed. Preferably the air evacuation unit connected includes an air inlet at one wall allowing entry of air into the interior of the treatment room and an air blower member mounted in a housing in the interior of the treatment room.

According to one important optional feature there is provided a sensor for detecting presence of a person inside the treatment room and a shut down system for shutting off all light fixtures in response to a signal indicative of a person in the treatment room.

According to one important optional feature there is provided there is provided a coating on interior surfaces of at least the side walls and roof arranged to prevent corrosion by the UVC light.

According to one important optional feature there is provided there is provided an insulating coating on the exterior of the treatment room. Preferably the exterior coating comprises a cork-based matrix.

According to one important optional feature there is provided the light fixtures at the roof and on the side walls form elongate florescent fixtures.

According to one important optional feature there is provided the light fixtures on the rack form LED strip lights. Preferably the LED strip lights are fastened to a horizontal shelf rail of the rack typically on an underside of each shelf with typically two rows of lights on each shelf.

According to one important optional feature there is provided there is provided a heater within the treatment room where the heater is arranged to heat the treatment room to a temperature in the range 90 to 160 degrees F.

According to one important optional feature there is provided the treatment room includes an end panel attached to the side walls including a single end door in the end panel.

According to one important optional feature there is provided there is provided a hanging rail attached to the roof along a center line of the treatment room arranged to receive hanging garments.

According to one important optional feature there is provided the rack has a plurality of shelves formed by front and rear rails with transverse wires on which articles to be sterilized can be suspended, the rails and wires being formed of stainless steel.

According to one important optional feature the shelves include at least one shelf which has an increased spacing from a next relative to other shelves.

According to one important optional feature the rack is mounted on rollers so as to be movable across the floor to different locations.

According to one important optional feature the light fixtures are mounted at the side walls comprise elongate members of for example 4 feet in length extending vertically along the side walls.

### BRIEF DESCRIPTION OF THE DRAWINGS

Embodiments of the invention will now be described in conjunction with the accompanying drawings in which:

FIG. 1 is a perspective view from one side with the adjacent side wall removed of an embodiment of a sterilization container according to the present invention.

FIG. 2 is a perspective view from end with the adjacent end wall removed of the embodiment of FIG. 1.

FIG. 3 is a top plan view with the roof removed of the embodiment of FIG. 1.

FIG. 4 is an end perspective view of the embodiment of FIG. 1.

FIG. 5 is a partial cross-section of the rack of FIG. 1.

### DETAILED DESCRIPTION

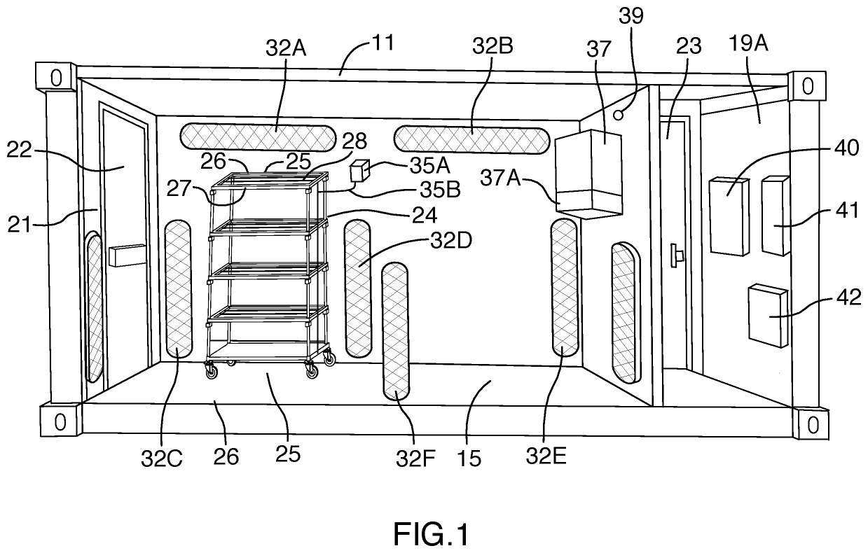

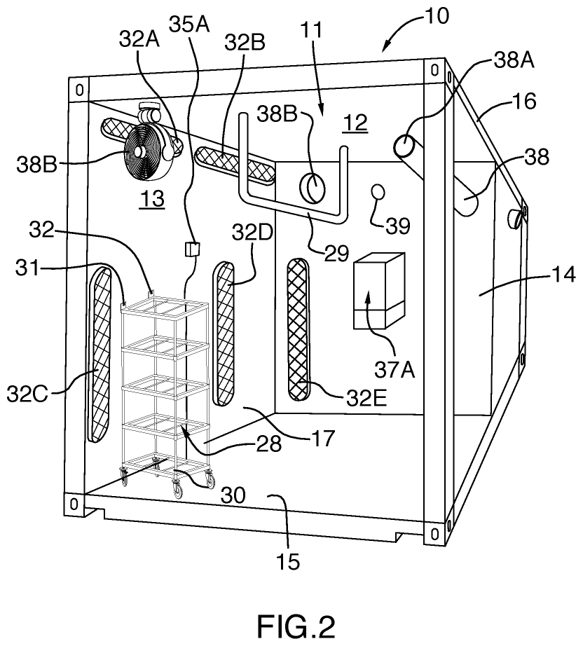

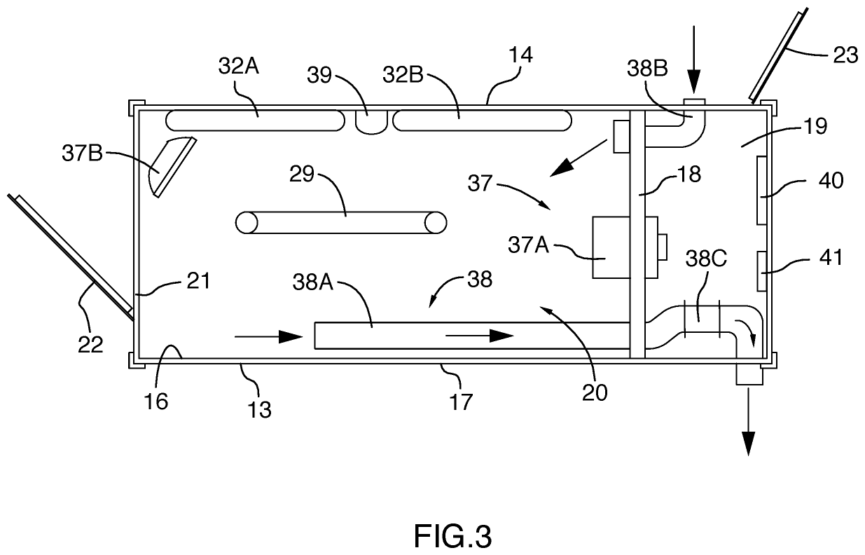

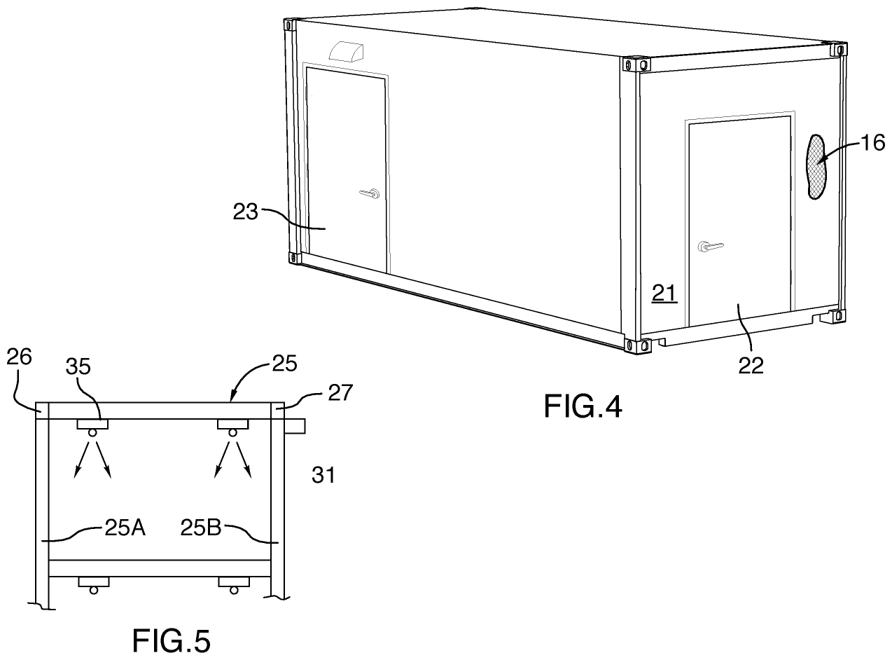

The portable container apparatus 10 of FIGS. 1 to 4 for use in sterilizing articles includes a portable shipping container 11 having a roof 12, two side walls 13, 14 and a floor 15 defining a portable structure formed of welded steel which can be lifted carried on a transport trailer.

There is provided a coating 17 on interior surfaces of at least the side walls and roof arranged to prevent corrosion by the UVC light. There is provided an insulating coating 16 on the exterior of the treatment room which can be formed of a cork-based matrix.

The container is divided by a closed separating wall 18 across the container from one side wall to the other and from the roof to the floor into a control room 19 at a first end having an end wall 19A and a treatment room 20 at a second end The treatment room 20 is closed at one end by a panel 21 which replaces the end doors of a conventional container and includes a central large door 22 of sufficient size to receive articles and racks including for example larger furniture such as a sofa which provides access to the treatment room. A door 23 in one side wall provides access to the control room.

Within the treatment room is provided a series of racks 24 each having a series of shelves 25 carried on posts 25A, 25B on which articles to be sterilized can be suspended.

Each rack has a plurality of shelves 25 formed by front and rear rails 26, 27 with transverse wires 28 on which articles to be sterilized can be suspended with the rails and wires being formed of stainless steel. The shelves can include a plurality of shelves at a common spacing and at least one shelf which has an increased spacing from a next relative to other shelves. The rack or each is mounted on rollers or castor wheels 30 so as to be movable across the floor to different locations. In this way the rack can be loaded outside the container and wheeled into place when the container comes available after a previous load.

The shelves can be used to received hanging articles attached by a separate hanger. items such as boots or skates can be mounted by inserting a top of the boot between two wires. The articles are mounted typically so that they are hung or draped and hence can move in an airflow. The the rack is removable for replacement by an alternative rack of different configuration or for a newly loaded rack.

The rack includes a first set of racks attached along one side wall and another set of racks attached along the second side wall leaving a central aisle for a person to enter to load the racks. the rack may extend along the full length or there may be provided racks end to end to fill the full length.

In order that the racks can be properly and readily removed and replaced, there are provided snap fasteners 31, 32 on the side wall for ready removal of the rack. The snap fasteners are typically provided at the top of the rack which is spaced from the roof so as to allow easy operation by the worker. in this way the rack is supported from the floor by the rollers and held against the wall by the fasteners allowing the worker to remove a filled and treated rack for replacement.

In addition a center hanging rail 29 is attached to the roof along a center line of the treatment room arranged to receive hanging garments such as medical gowns which are thus located individually on the center rail along the center aisle.

In order to provide sterilization treatment of the articles on the racks and the rail, there is provided a plurality of light fixtures 32A to 32F arranged at the top of the side wall and at spaced positions along the height of the side wall. Thus each of the light fixtures on the walls and optionally the roof are elongate florescent type fittings typically of a length of the order of 4 feet and designed to accommodate high ambient heat. The fittings at or on the roof extend longitudinally of the treatment room so as to direct light downwardly on to the top of the ach rack and into the interiors of the articles on the racks. The fixtures on the walls stand vertically and are located between the ends of two adjacent racks so as to direct light against the end face of the rack and again into the interior of the racks and the articles thereon. The light fixtures are arranged to generate UVC illumination at a wavelength and intensity sufficient to kill biological material such as viruses within a suitable time period.

That is, some of the light fixtures are mounted at the roof for illuminating downwardly onto the articles on the rack and some of the light fixtures are mounted at the side walls for illuminating inwardly into the interior of the treatment room onto the articles on the rack.

In addition each rack at least one of the light fixtures mounted on the rack. Thus as shown in FIG. 5, the racks have LED light strips 35 mounted on each shelf on the underside along the length of the rack to direct UVC light from the LEDs downwardly onto the next self and into the articles on the shelf or suspended between the shelves. Preferably two strips of LEDs are provided on each shelf. The light strips are connected to a common coupling 35B which is attached as a common connection to a rack supply receptacle 35A on the side wall.

In order to ensure that all articles are properly irradiated on all surfaces there is provided an airflow generating fan construction 37 arranged to generate a circulation of air within the treatment room so as to cause movement by the air of the articles on the racks to ensure that the UVC illumination reaches all surfaces of the articles.

The fan construction 37 includes a first heater fan 37A on the divider wall 18 to generated a heated airflow and a second airflow and a second air movement fan 37B at one corner between the side wall and an end wall fan an angle to the first fan so as to cause a circulation of the air within the treatment room to generate turbulence and swirling which tends to move or flutter the articles to move its surfaces to allow penetration of the illumination.

There is also provided an air evacuation unit 38 connected between the interior of the treatment room and the exterior to rapidly remove heated air in the treatment room after the treatment is completed. This includes an air inlet duct 38B at one wall allowing entry of air into the interior of the treatment room and an air outlet blower duct member 38A mounted in a housing in the interior of the treatment room connecting to a blower 38C which acts to rapidly change the air from the interior to the exterior.

Further there is provided a sensor 39 for detecting presence of a person inside the treatment room and a shut down system 40 forming part of a control system including control components 41 and 42 on the end wall 19A for shutting off all light fixtures in response to a signal indicative of a person in the treatment room.

The heater 37A is arranged to heat the treatment room to a temperature in the range 90 to 160 degrees F. This temperature is not intended to be sufficient to kill insects but is effective with the UV light in treating biological material.

1. A portable container apparatus for use in sterilizing articles which could be infected with a pathogen comprising:
a portable shipping container having a roof, two side walls and a floor defining a portable structure which can be carried on a transport trailer;
the container being divided by a separating wall across the container into a control room at a first end and a treatment room at a second end;
at least one door providing access to the treatment room and at least one door providing access to the control room;
at least one rack having a plurality of rails on which articles to be sterilized can be suspended;
a plurality of light fixtures each arranged to generate UVC illumination;
some of the light fixtures being mounted at the roof for illuminating downwardly onto the articles on the rack;
some of the light fixtures being mounted at the side walls for illuminating inwardly into the interior of the treatment room onto the articles on the rack;
at least one of the light fixtures being mounted on the rack;
and an airflow generating fan construction arranged to generate a circulation of air within the treatment room so as to cause movement by the air of the articles on the rack to ensure that the UVC illumination reaches all surfaces of the articles.

2. The apparatus according to claim 1 wherein there is provided a rack at each side wall with a center aisle between the racks.

3. The apparatus according to claim 1 wherein the rack is removable for replacement by an alternative rack of different configuration and includes an electrical coupling connecting with each light fixture on the rack for common attachment to an outlet in the treatment room.

4. The apparatus according to claim 1 wherein the rack is mounted on the side wall.

5. The apparatus according to claim 1 wherein there are provided snap fasteners on the side wall for ready removal of the rack.

6. The apparatus according to claim 1 wherein the rack includes a plurality of shelves.

7. The apparatus according to claim 1 wherein the fan construction includes a first heater fan and a second airflow fan at an angle to the first fan so as to cause a circulation of the air.

8. The apparatus according to claim 7 wherein the first heater fan is arranged at the divider wall and the second airflow fan ais arranged at a corner between a side wall and an end wall.

9. The apparatus according to claim 1 wherein there is provided an air evacuation unit connected between the interior of the treatment room and the exterior to rapidly remove heated air in the treatment room after the treatment is completed.

10. The apparatus according to claim 9 wherein the air evacuation unit connected includes an air inlet at one wall allowing entry of air into the interior of the treatment room and an air blower member mounted in a housing in the interior of the treatment room.

11. The apparatus according to claim 1 including a sensor for detecting presence of a person inside the treatment room and a shut down system for shutting off all light fixtures n response to a signal indicative of a person in the treatment room.

12. The apparatus according to claim 1 wherein there is provided a coating on interior surfaces of at least the side walls and roof arranged to prevent corrosion by the UVC light.

13. The apparatus according to claim 1 wherein there is provided an insulating coating on the exterior of the treatment room.

14. The apparatus according to claim 1 wherein the light fixtures at the roof and on the side walls form elongate florescent fixtures.

15. The apparatus according to claim 1 wherein the light fixtures on the rack form LED strip lights.

16. The apparatus according to claim 16 wherein the LED strip lights are fastened to a horizontal shelf rail of the rack.

17. The apparatus according to claim 1 wherein there is provided a heater within the treatment room.

18. The apparatus according to claim 1 wherein the treatment room includes an end panel attached to the side walls including a single end door in the end panel.

19. The apparatus according to claim 1 wherein there is provided a hanging rail attached to the roof along a center line of the treatment room arranged to receive hanging garments.

20. The apparatus according to claim 1 wherein the rack has a plurality of shelves formed by front and rear rails with transverse wires on which articles to be sterilized can be suspended, the rails and wires being formed on stainless steel.

21. The apparatus according to claim 1 wherein the rack is mounted on rollers so as to be movable across the floor to different locations.
